(19) United States
(12) Patent Application Publication
(10) Pub. No.: US 2022/0248644 A1
(43) Pub. Date: Aug. 11, 2022

(54) METHOD AND COMPOSITION FOR IMPROVING HEALTH OF AN AVIAN

(71) Applicant: SUPERBREWED FOOD, INC., New Castle, DE (US)

(72) Inventors: Alon KARPOL, Tel Mond (IL); Hadar GILARY, Magal (IL); Shawn William JONES, Bear, DE (US); Bryan P. TRACY, Wilmington, DE (US); Biniam MARU, Bear, DE (US); Aharon M. EYAL, Jerusalem (IL); Sasson SOMEKH, Los Altos Hills, CA (US)

(21) Appl. No.: 17/613,517

(22) PCT Filed: May 22, 2020
(86) PCT No.: PCT/IB2020/054873

§ 371 Date: Nov. 23, 2021

Related U.S. Application Data
(60) Provisional application No. 62/852,326, filed on May 24, 2019.

(51) Int. Cl. A01K 45/00

(52) U.S. Cl. CPC A01K 45/007

(57) ABSTRACT

Provided are methods and compositions an organism of the order Clostridiales for improving the health of an avian, wherein an effective dose of the composition is applied to an outer surface of an egg containing the avian as an embryo prior to hatching. Also disclosed are compositions comprising a strain within the order Clostridiales selected from the group consisting of Caldicoprobacteraceae; Christensenellaceae; Clostridiaceae; Defluviitaleaceae; Eubacteriaceae; Graciibacteraceae; Heliobacteriaceae; Lachnospiraceae; Oscillospiraceae; Peptococcaceae; Peptostreptococcaceae; Ruminococcaceae; Syntrophomonadaceae and combinations thereof and their use in such methods.

### CROSS REFERENCE TO RELATED APPLICATION

The instant application claims priority to PCT/IB2020/054873, filed 22 May 2020, which in turn claims priority to U.S. Provisional Application No. 62/852,326 filed 24 May 2019, the disclosure of which is incorporated by reference herein in its entirety.

### FIELD OF THE INVENTION

The field of art to which this invention generally pertains is improving health of an animal and more specifically to methods of use of compositions comprising cells of organism of the order Clostridiales for improving health of an animal.

### BACKGROUND OF THE INVENTION

A number of documents have appeared in the literature describing the scientific basis for use of probiotics, as intestinal inoculants for animals. Since Metchnikoffs work in the 19^{th} century, which first established the probiotic understanding as we know it, many studies have shown the ability of microorganisms to suppress pathogen growth, improve feed conversion ratio or stimulate the immune system. For example, feeding viable Lactobacillus acidophilus cells to young dairy calves was shown to reduce the incidence of diarrhea, and increase the numbers of lactobacilli and reduce coliform counts in feces.

It is generally held that during periods of low disease resistance, such as stress, undesirable microorganisms are able to proliferate in the gastrointestinal tract (GI tract) of animals, humans included. Maintaining a normal, healthy balance of microorganisms is deemed to be critical, particularly during such stressful periods. The concept underlying use of probiotics therefore is that, if sufficient numbers of an appropriate microorganism(s) are introduced into the intestinal tract (i) at times of stress and/or disease, (ii) at birth, or (iii) after antibiotic treatment, the negative consequences of the microbial imbalances (Dysbiosis) can be minimized or overcome. Using such preparations of live, naturally occurring microorganisms helps restore and maintain the proper balance of beneficial microbes in the GI tract during times of stress, disease, and following antibiotic therapy.

Probiotics for avian species are bacterial or yeast preparations that are administered orally or added to feeds. Oral administration is a relatively simple process to execute, but the microorganisms must survive the passage in the stomach at low pH and to show tolerance to the bile salts in the GI tracks, until they colonize the intestinal tracks. Another method to administer probiotics is by ovo injection. In this process, unhatched eggs are injected with a probiotic, so that the colonization is done at an early stage of the life cycle of the chick, when the GI tract is not inhabited by other bacteria. However, by their very nature, in ovo injections cause a breakage in the eggshell which exposes the egg to potential contamination risks. This is a major concern in the industry such that both before and after the injection, the egg surface is thoroughly cleaned to reduce any pathogen exposure. Additionally, in ovo injection requires specialized equipment. An alternative to these other administration methods is spraying unhatched eggs with the agent (bacteria, yeast) before it hatches. The eggshell itself is permeable, allowing oxygen transfer necessary for the developing chick, and bacteria are known to be able to penetrate the eggshell. The most general mechanism of penetration is a temperature differential between the egg and any liquid on its surface. If the egg is warmer than the liquid on its surface or the ambient air, it begins to cool. As the egg cools, the contents retract producing a negative pressure within the egg. Any bacteria on the egg's surface are then pulled through the eggshell and its membranes. The most common area of penetration is the air cell (i.e., the blunt end of the egg), though any part can be penetrated.

There are several advantages to spraying over other application methods. First, like in ovo injection, probiotics are introduced to the developing embryo at a very early stage and can improve GI colonization. However, unlike in ovo injections, spraying does not break the eggshell and thus can significantly reduce the risk of pathogen exposure. Second, administration is simple and straightforward without needing specialty equipment and can be applied multiple times during incubation or even storage of the eggs.

### SUMMARY OF THE INVENTION

According to an aspect of some embodiments of the present invention, provided is a method of improving health of an avian comprising providing a composition comprising an organism of the order Clostridiales, and applying an effective dose of said composition to an outer surface of an egg containing the avian as an embryo prior to hatching, thereby improving said avian health.

### DETAILED DESCRIPTION OF THE INVENTION

The present invention relates to methods of use of compositions comprising an organism of the order Clostridiales for improving the health of an animal.

In today's practice, the main bacteria that are being administered to eggs to improve the health of an avian are genera such as Lactobacillus, Enterococcus, and Bifidobacterium. Although those genera are beneficial in some cases, they do not produce detectable level of butyric acid, if at all, which is an essential molecule to stimulate the GI tract activity against pathogens.

It has been well documented that facultative anaerobes, particularly pathogens like Salmonella, can populate the eggshell surface (which is an aerobic environment), penetrate the eggshell, and then propagate in the anaerobic egg interior.

The present inventors have surprisingly found that strict anaerobes such as Clostridiales can survive on an eggshell surface long enough to penetrate into the low-oxygen environment of the egg interior, which is unexpected in view of the fact that the egg must remain in an aerobic environment for the developing embryo.

The particulars shown herein are by way of example and for purposes of illustrative discussion of the various embodiments of the present invention only and are presented in the cause of providing what is believed to be the most useful and readily understood description of the principles and conceptual aspects of the invention. In this regard, no attempt is made to show details of the invention in more detail than is necessary for a fundamental understanding of the invention, the description making apparent to those skilled in the art how the several forms of the invention may be embodied in practice.

The present invention will now be described by reference to more detailed embodiments. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention. As used in the description of the invention and the appended claims, the singular forms “a,” “an,” and “the” are intended to include the plural forms as well, unless the context clearly indicates otherwise.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Every numerical range given throughout this specification will include every narrower numerical range that falls within such broader numerical range, as if such narrower numerical ranges were all expressly written herein.

Additional advantages of the invention will be set forth in part in the description which follows, and in part will be obvious from the description, or may be learned by practice of the invention. It is to be understood that both the foregoing general description and the following detailed description are exemplary and explanatory only and are not restrictive of the invention, as claimed.

As used herein, the term “improving health” is intended to mean regulating a system of the body, such as by treating, preventing or reducing incidence and/or severity and/or duration of known conditions affecting an avian as compared to the incidence and/or severity of such conditions in the absence of use of the method as disclosed herein.

According to some embodiments, “reducing incidence” refers to a reduction of at least 20% in the number of occurrences of at least one symptom of the condition over a given time period. According to some embodiments, “reducing severity” refers to a reduction of at least 20% in the severity of least one symptom of the condition over a given time period. According to some embodiments, “reducing duration” refers to a reduction of at least 20% of the duration of at least one symptom of the condition at above a defined threshold level.

As used herein, the term “colonizing” with regard to a digestive tract is intended to mean populating the digestive tract with at least 10\hat{ }2 CFU of bacteria per 1 g.

As used herein, the term “regulating the immune system” is intended to mean stimulating or inhibiting activity of the immune system as appropriate for improving the health of the avian, such as by increasing immune activity against a pathogen.

As used herein, the term “improving digestion” is intended to mean an increase in nutrient absorption by at least 5% compared to a group not provided with the invention. In some embodiments, nutrient absorption is determined by measuring the total nutrient input and non-absorbed nutrients in the feces.

As used herein, each of the terms, “inhibiting the growth of pathogenic bacteria”, “reducing or preventing respiratory problems”, “reducing or preventing Coccidia infection”, “reducing or preventing dysbiosis”, “reducing or eliminating antibiotic use”, “decreasing mortality”, and “reducing deformities” refers to a reduction of at least 10% as compared to the occurrence of the specified parameter in the absence of use of the compositions as disclosed herein.

As used herein, the term “improved hatchability” refers to an increase of at least 5% in the number of eggs hatching in a batch of at least 100 eggs.

As used herein, the term “feed utilization” refers to an amount of feed consumed by an animal per day on a moisture-free basis. As used herein, the term “improving feed utilization” refers to increasing feed utilization by at least 5%, such as by at least 5%, by at least 10% or even by at least 15%.

As used herein, the term “feed conversion” refers to feed dry matter intake in kilograms per kilogram weight gain of an animal.

As used herein, the term “improving feed conversion” refers to increasing feed conversion by at least 5%, by at least 10% or even by at least 15%.

As used herein, the term “tolerance to pH” refers to survival of at least 1% of the cells exposed to the condition for duration of 2 hours.

As used herein, the term “tolerance to bile salts” with regard to cells refers to survival of at least 10% of the cells when exposed to a solution comprising least 0.2 wt % bile salts, at least 1 wt % bile salts or even at least 2 wt % bile salts for a duration of 24 hours.

According to an aspect of some embodiments of the present invention, provided is a method of improving health of an avian comprising providing a composition comprising an organism of the order Clostridiales, and applying an effective dose of said composition to an outer surface of an egg containing the avian as an embryo prior to hatching, thereby improving said avian health.

According to an aspect of some embodiments of the present invention, there is provided a composition comprising an organism of the order Clostridiales for use in treating an avian, wherein said composition is for applying to an outer surface of an egg containing the avian as an embryo prior to hatching.

According to an embodiment, said applying comprises spraying.

Spraying may be carried out according to any method known in the art, including high pressure spraying, low pressure spraying and electrostatic spraying.

According to an embodiment, said applying comprises electrostatic spraying.

According to an embodiment, said egg has a narrow end and a blunt end, wherein said applying comprises spraying the narrow end of the egg.

According to an embodiment, the amount of composition applied per unit area on the narrow end of the egg is greater than the amount of composition applied per unit area on the blunt end of the egg, such as at least 10 wt %, at least 15%, at least 20%, at least 25%, at least 30%, at least 35%, at least 40%, at least 45%, at least 50%, at least 55%, at least 60%, at least 65%, at least 70%, at least 75% or even at least 80% greater.

According to an embodiment, said providing said composition comprises mixing said organism with a carrier to produce a mixture and adjusting said mixture to provide anaerobic conditions.

According to an embodiment, adjusting said mixture comprises adding at least one oxygen scavenger/reducing agent, such as one selected from the group consisting of cysteine hydrochloride, sodium sulfide, sodium sulfite, sodium metabisulfite and combinations thereof.

According to an embodiment, oxygen concentration in said composition is in the range of from about 1 microMolar to about 3000 mMolar. In some embodiments the oxygen concentration is less than 100 microMolar, less than 50 microMolar, less than 10 micorMolar or even less than 1 microMolar.

According to an embodiment, said providing said composition further comprises adding a reducing agent to said mixture. According to an embodiment, said reducing agent is selected from the group consisting of cysteine hydrochloride, sodium sulfide, sodium sulfite, sodium metabisulfite and combinations thereof.

According to an embodiment, said providing said composition further comprises cooling said mixture. According to an embodiment, cooling said mixture comprises passing said mixture through cold water or a cold gas such as nitrogen.

According to an embodiment, said composition comprises at least 10⁴ colony forming units per milliliter of said organism, such as at least 10⁵, at least 10⁶, at least 10⁷, at least 10⁸, at least 10⁹ or up to 10¹⁰ CFU per milliliter of said organism.

According to an embodiment, a concentration of said organism in said composition, a temperature of said composition and an air content of said composition are each adjusted to provide at least 10² CFU per cm² eggshell of said organism within said egg, such as at least 10³, at least 10⁴, at least 10⁵, at least 10⁶, at least 10⁷ or up to 10⁸ CFU per cm² eggshell of said organism within said egg.

According to an embodiment the carrier is selected from the group consisting of aqueous solution of salts and/or of sugars. According to an embodiment the sugars are selected from the group consisting of mannitol, lactose, cellulose, glucose, sucrose, starch, amylose, fructose, and fructose oligo saccharides (FOS). According to an embodiment the salts are selected from the group consisting of NaCl, CaCl2, MgCl2, and tris(hydroxymethyl)aminomethane. According to an embodiment the concentration of the salt ranges from 0.05% to 0.5% of the solution. According to an embodiment the concentration of the sugar ranges from 2% to 10% of the solution. According to an embodiment the carriers are selected from a group consisting of antimicrobials, antioxidants, chelating agents, inert gases, organic acids, glycol, polyethylene glycol, vegetable oils, ethyl oleate. According to an embodiment the carriers are phosphate buffer solution or glycerol solution.

According to an embodiment, applying is carried out at least twice, such as at least three times, at least four times or even at least five times.

According to an embodiment, said administering results in at least 10 organism Colony-Forming Units per gram wet feces of said avian on the second day after hatching. According to an embodiment, said administering results in up to 10₁₀ organism Colony-Forming Units per gram wet feces of said avian on the second day after hatching.

According to an embodiment, said administering results in at least 100 Colony-Forming Units per gram wet feces of said avian on the twentieth day after hatching. According to an embodiment, said administering results in up to 10₁₁ organism Colony-Forming Units per gram wet feces of said avian on the second day after hatching.

According to an embodiment said organism is characterized by having at least one property selected from the group consisting of butanoate metabolism, obligate anaerobic growth, gas fixation via the reductive acetyl-coenzyme A pathway, tolerance to bile salts at concentration greater than 0.05%, greater than 0.1% greater than 0.2% or greater than 0.4%, tolerance to pH of less than 5.5, less than 4.5 less than 3.5 or less than 2.5, and self-aggregation.

According to an embodiment, said organism is selected from the group consisting of Caldicoprobacteraceae; Christensenellaceae; Clostridiaceae; Defluviitaleaceae; Eubacteriaceae; Graciibacteraceae; Heliobacteriaceae; Lachnospiraceae; Oscillospiraceae; Peptococcaceae; Peptostreptococcaceae; Ruminococcaceae; Syntrophomonadaceae and combinations thereof.

According to an embodiment, said organism is Eubacterium aggregans.

According to an embodiment, said organism comprises a member of a genus selected from the group consisting of Acetobacterium, Acetoanaerobium, Blautia, Butyribacterium, Clostridium, Desulfitobacterium, Desulgotomaculum, Eubacterium, Hungateiclostridium, Lachnoclostridium, Moorella, Oxobacter, Paraclostridium Peptoclostridium, Pseudoclostridium, Ruminiclostridium, Sporomua, Terrisporobacter, Thermoanaerobacter, Thermoanaerobacterium, Thermoclostridium and combinations thereof.

According to an embodiment, said organism comprises at least one selected from the group consisting of Acetobacterium woodii, Blautia producta, Butyribacterium methylotrophicum, Clostridium acetobutylicum, Clostridium autoethanogenum, Clostridium beijerinckii, Clostridium butyricum, Clostridium carboxidivorans, Clostridium drakei, Clostridium ljungdahlii, Clostridium kluyveri, Clostridium pasteurianum, Clostridium saccharobutylicum, Clostridium saccharoperbutylacetonicum, Clostridium scatologenes, Clostridium tyrobutyricum, Eubacterium aggregans, Eubacterium limosum, Eubacterium callendari, Eubacterium hallii, Eubacterium maltosivorans, Paraclostridium bifermentans, Oxobacter pfennigii, Sporomusa termitida, and Terrisporobacter glycolicus.

According to an embodiment the avian is selected from the group consisting of broilers, hens, turkeys, ducks, and fowls.

According to an embodiment, said improving health comprises colonizing a digestive track of said avian with said organism.

According to an embodiment, said improving health comprises colonizing a digestive track of said embryo with said organism.

According to an embodiment, said colonizing comprises colonizing at least one selected from the group consisting of duodenum, jejunum, ileum, small intestine, cecum, and colon of said avian.

According to an embodiment, said improving health comprises at least one selected from the group consisting of regulating the immune system, improving digestion, inhibiting the growth of pathogenic bacteria, forming butyric acid, reducing or preventing respiratory problems, reducing or preventing Coccidia infection, utilizing lactic acid, reducing or preventing dysbiosis, improving feed utilization, improving feed conversion, reducing or eliminating antibiotic use, decreasing mortality, and reducing deformities.

According to an embodiment, said rate of forming butyric acid is at least 0.01 millimole per hour five days after hatching, such as at least 0.5 mM, at least 1.0 mM or at least 2 mM per hour.

According to an embodiment the improving health comprises forming five days after hatching butyric acid at a rate of at least 0.005 millimole per hour, at least 0.01 millimole per hour, at least 0.05 millimole per hour, at least 0.1 millimole per hour.

According to an embodiment, said composition comprises a live culture of said organism.

According to an embodiment, said composition comprises a sporulated culture of said organism.

According to an embodiment the composition further comprises at least one of water, nutrients, prebiotics, and probiotics. According to an embodiment the prebiotics comprise at least one selected from the group consisting of trans-galactooligosaccharide, inulin, resistance starch, pectin, beta glucans and combinations thereof.

According to an embodiment, said composition further comprises cells of at least one selected from the group consisting of Bacillus amyloliquefaciens; BacillusBacillus toyonensis; BacillusBacillus coagulans; BacillusBacillus licheniformis; Bacillus megaterium; Bacillus mesentricus; Bacillus polymyxa; Bacillus subtilis; Bifidobacterium animalis; Bifidobacterium bifidium; Bifidobacterium bifidus; Bifidobacterium thermophilus; Bifidobacterium longum; Bifidobacterium pseudolongum; Bifidobacterium lactis; Clostridium butyricum; Enterococcus faecium; Enterococcus faecalis; Escherichia coli; Lactobacillus thermophilus; Lactobacillus acidophilus; Lactobacillus brevis; Lactobacillus bulgaricus; Lactobacillus casei; Lactobacillus delbrueckii; Lactobacillus subspecies; Lactobacillus bulgaricus; Lactobacillus farciminis; Lactobacillus fermentum; Lactobacillus gallinarum; Lactobacillus jensenii; Lactobacillus paracasei; Lactobacillus plantarum; Lactobacillus reuteri; Lactobacillus rhamnosus; Lactobacillus lactis; Lactobacillus salivarius; Lactobacillus sobrius; Megasphaera elsdenii; Pediococcus acidolactici; Propionibacterium shermanii; Propionibacterium freudenreichii; Propionibacterium acidipropionici; Propionibacterium jensenii; Saccharomyces bourlrdii; Saccharomyces cerevisiae; Saccharomyces servisia; Streptococcus faecalis; Streptococcus faecium; Streptococcus gallolyticus; Streptococcus salivarius; Streptococcus subsp.; Streptococcus thermophilus; Streptococcus bovis and combinations thereof.

According to an embodiment, said composition comprises a mixture of organisms. According to an embodiment, said mixture of organisms is a syntrophic mixture showing a syntrophic behavior. According to an embodiment, said syntrophic behavior is beneficial to said animal. According to an embodiment, said mixture of organisms comprises at least one CO₂-utilizing organism. According to an embodiment, said CO₂-utilizing organism is an acetogen. According to an embodiment, said acetogen is selected from the group consisting of Acetitomaculum; Acetoanaerobium; Acetobacterium; Acetohalobium; Acetoneme; Bacillus; Blautia; Bryantella; Butyribacterium; Caloramator; Clostridium; Desulfovibrio; Entrococcus; Eubacterium; Gottschalkia; Holophage; Methylobacterium; Micrococcus; Moorella; Mycobacterium; Natronielle; Natronincola; Oxobacter; Peptoniphilus; Proteus; Reticulitermes; Rhizobium; Ruminococcus; Saccharomyces; Sinorhizobium; Sphingomonas; Sporomusa; Syntrophococcus; Thermoacetogenium; Tindallia; Treponema; Veillonella and combinations thereof.

According to an embodiment, said mixture of organisms comprises at least one non-CO₂ utilizing organism. According to an embodiment, said mixture of organisms comprises at least one acetate-forming organism and at least one acetate-utilizing organism. According to an embodiment, said mixture of organisms comprises at least one lactate-forming organism and at least one lactate-utilizing organism.

According to an embodiment, said applying improves hatchability of eggs.

According to an embodiment, said applying reduces the time until hatching.

According to an embodiment, said applying allows eggs to be stored for greater than two weeks with less than 15% reduction in the hatchability.

According to one aspect of the current invention, provided is a method for preparing the composition, which method comprises anaerobic fermentation, induction of sporulation and separation of the formed cells. According to an embodiment the method for induction of sporulation comprises cultivating at phosphate concentration of less than 5%, less than 3% or less than 1%. According to an embodiment, the induction of sporulation comprises cultivating at nitrogen concentration of less than 1 mM, less than 0.1 mM or less than 0.01 mM. According to an embodiment, the induction of sporulation comprises cultivating at a temperature greater than 37 degree C., greater than 55 degree C. or greater than 75 degree C. According to an embodiment, the induction of sporulation comprises cultivating at a temperature of less than 37 degree C., less than 25 degree C. or less than 10 degree C. According to an embodiment, the induction of sporulation comprises reducing or preventing respiratory problems, reducing or preventing Coccidia infection in a medium comprising at least 100 mM solvent, at least 10 mM or at least 1 mM. According to an embodiment the method for induction of sporulation comprises cultivating at carbon concentration of less than 10 mM, less than 1 mM or less than 0.1 mM. According to an embodiment the method for induction of sporulation comprises cultivating at pH of less than 6.0, less than 5.0 or less than 4.0. According to an embodiment the method for induction of sporulation comprises cultivating at pH of more than 8.0, more than 9.0 or more than 10.0.

According to an embodiment, the separating of cells comprises at least one of flocculating, centrifuging and separating by large scale flow cytometry, to separate the vegetative cells from the spores.

### EXAMPLES

### Example 1—Tolerance of E. aggregans to Acidic Conditions and to Bile Salts

Method

A single bacterial colony was introduced into 10-ml Reinforced Clostridium Media (RCM) medium, under anaerobic conditions, to serve as an inoculum. The bacteria were cultivated overnight at 37 degree C. RCM solutions of pH 4.5 and RCM solutions comprising a desired concentration of bile salts (Oxgal) in the range of 0.2 to 1.0 wt % were prepared.

The medium was filtered into 10-ml tubes (8-ml medium in each tube) and deoxygenated in an anaerobic chamber overnight.

The 10-ml tubes comprising medium of desired pH or desired bile salts concentration were inoculated with 80 microliter of inoculum culture and incubated at 37 C.° for 2 hours or for 48 hours (for low pH and bile acid tolerance experiences, respectively). The bacterial cells were diluted by a factor of ×100 in RCM and plated on RCM agar plates. The cells were cultivated on the plates for 48 hours at 37 degree C. and the number of Colony Forming Units (CFU) were counted.

Survival rate was determined by dividing the number of cells on the plates by the number of cells that were challenged at pH 6.8 or no bile salts (for low pH and bile acid tolerance experiences, respectively).

Tolerance was identified as 45% survival at 0.2% bile salts concentration and 20% survival between 0.5% to 2.0% bile salts concentrations.

Similarly, E. aggregans were contacted under anaerobic conditions for 2 hours with RCM solutions of acidic pH. There was 77% survival at pH 4.5. These results demonstrate the tolerance in anaerobic conditions of E. aggregans to acidic pH and to a relatively high concentration of bile salts.

### Example 2—Bacterial Aggregation of E. aggregans

Aggregation characteristics for E. aggregans were measured as follows.

A single colony of E. aggregans was inoculated in 10-ml of RCM medium. The cells were grown in an anaerobic chamber at 37 C.° for 1-2 days. When the cells reached stationary growth phase, the cells were centrifuged at 3000 rpm for 5 minutes. The supernatant was removed and the pellet obtained was re-suspended in 10-ml PBS. 4 ml of cells resuspended in PBS were mixed with 4 ml of fresh growth medium (RCM) and the Absorbance Unit (AU) was adjusted to 1 by addition of PBS. The mixture was again mixed and an initial absorbance value was taken (ODto). Percent aggregation capacity (% AC) value was calculated by % AC=(1−(OD_{tf}/ODₜₒ)/100, where OD_{tf} and ODₜₒ are the optical density at final and initial times, respectively.

The results show that after 5 hours more than 85% of the cells have aggregated. This result confirms the ability of E. aggregans to aggregate quickly. As such during the passage of the cells through the GI tracks, the cells have a better chance to adhere to the epithelial cells of the animal and colonize them.

### Example 3—Butyrate Production by E. aggregans

The ability of E. aggregans, in conditions mimicking the environment of the GI tracks, to produce butyric acid and to consume lactic acid was measured as follows. The result showed that 94% of the lactic acid was consumed after 4 days and that butyric acid concentration reached 64 mM.

A mid-log culture of E. aggregans was inoculated into 50-ml RCM medium, supplemented with 50 mM of lactic acid. The fermentation took place in serum bottles at 37 C without shaking. The RCM medium contains 0.5% glucose, 0.22% acetic acid. Samples were removed after 24 hours and 96 hours for HPLC analysis.

These results confirmed the viability of E. aggregans at conditions that are expected to be encountered in the GI tracks of the animal or human to produce butyric acid, a known chemical that helps the immune system of the animal to fight pathogens. One of the carbon source for butyric acid production is the prevailing molecule of lactic acid in the GI tracks. Accordingly, the lactic acid was consumed almost completely by the cells of E. aggregans.

1. A method of improving health of an avian comprising providing a composition comprising an organism of the order Clostridiales and spraying an effective dose of said composition onto an outer surface of an egg containing the avian as an embryo prior to hatching, thereby improving said avian health.

2-3. (canceled)

4. The method of claim 1, said egg having a narrow end and a blunt end, wherein said spraying is applied to the narrow end of the egg.

5. (canceled)

6. The method of claim 1, wherein said providing said composition comprises mixing said organism with a carrier to produce a mixture and adjusting said mixture to provide anaerobic conditions.

7. The method of claim 1, wherein an oxygen concentration in said composition is less than 100 microMolar.

8. The method of claim 6, wherein said providing said composition further comprises adding a reducing agent to said mixture.

9. (canceled)

10. The method of claim 1, wherein said composition comprises at least 10⁴ colony forming units per milliliter of said organism.

11. The method of claim 1, wherein a concentration of said organism in said composition, a temperature of said composition and an air content of said composition are each adjusted to provide at least 10² colony forming units per cm² eggshell of said organism within said egg.

12-13. (canceled)

14. The method of claim 1, wherein said spraying results in at least 100 Colony-Forming Units per gram wet feces of said avian on a twentieth day after hatching.

15. The method of claim 1, wherein said organism is characterized by having at least one property selected from the group consisting of butanoate metabolism, obligate anaerobic growth, gas fixation via the reductive acetyl-coenzyme A pathway, tolerance to bile salts at concentration greater than 0.05%, tolerance to pH of less than 3.5, and self-aggregation.

16. The method of claim 1, wherein said organism is selected from the group consisting of Caldicoprobacteraceae; Christensenellaceae; Clostridiaceae; Defluviitaleaceae; Eubacteriaceae; Graciibacteraceae; Heliobacteriaceae; Lachnospiraceae; Oscillospiraceae; Peptococcaceae; Peptostreptococcaceae; Ruminococcaceae; Syntrophomonadaceae and combinations thereof.

17. The method of claim 1, wherein said organism is Eubacterium aggregans.

18. The method of claim 1 wherein said organism comprises a member of a genus selected from the group consisting of Acetobacterium, Acetoanaerobium, Blautia, Butyribacterium, Clostridium, Desulfitobacterium, Desulgotomaculum, Eubacterium, Hungateiclostridium, Lachnoclostridium, Moorella, Oxobacter, Paraclostridium Peptoclostridium, Pseudoclostridium, Ruminiclostridium, Sporomua, Terrisporobacter, Thermoanaerobacter, Thermoanaerobacterium, Thermoclostridium and combinations thereof.

19. The method of claim 1 wherein said organism is selected from the group consisting of Acetobacterium woodii, Blautia producta, Butyribacterium methylotrophicum, Clostridium acetobutylicum, Clostridium autoethanogenum, Clostridium beijerinckii, Clostridium butyricum, Clostridium carboxidivorans, Clostridium drakei, Clostridium ljungdahlii, Clostridium kluyveri, Clostridium pasteurianum, Clostridium saccharobutylicum, Clostridium saccharoperbutylacetonicum, Clostridium scatologenes, Clostridium tyrobutyricum, Eubacterium aggregans, Eubacterium limosum, Eubacterium callendari, Eubacterium hallii, Eubacterium maltosivorans, Paraclostridium bifermentans, Oxobacter pfennigii, Sporomusa termitida, Terrisporobacter glycolicus and combinations thereof.

20-23. (canceled)

24. The method of claim 1, wherein said improving health comprises at least one selected from the group consisting of regulating the immune system, improving digestion, inhibiting the growth of pathogenic bacteria, forming butyric acid, reducing or preventing respiratory problems, reducing or preventing Coccidia infection, utilizing lactic acid, reducing or preventing dysbiosis, improving feed utilization, improving feed conversion, reducing or eliminating antibiotic use, decreasing mortality, and reducing deformities.

25. The method of claim 1 wherein said improving health comprises forming butyric acid at a rate sufficient to reach butyric acid concentration of at least 0.1 millimolar in the digestive track five days after hatching.

26-27. (canceled)

28. The method of claim 1, wherein said composition comprises a sporulated culture of said organism.

29. (canceled)

30. The method of claim 1, wherein said composition further comprises cells of at least one selected from the group consisting of Bacillus amyloliquefaciens; BacillusBacillus toyonensis; BacillusBacillus coagulans; BacillusBacillus licheniformis; Bacillus megaterium; Bacillus mesentricus; Bacillus polymyxa; Bacillus subtilis; Bifidobacterium animalis; Bifidobacterium bifidium; Bifidobacterium bifidus; Bifidobacterium thermophilus; Bifidobacterium longum; Bifidobacterium pseudolongum; Bifidobacterium lactis; Clostridium butyricum; Enterococcus faecium; Enterococcus faecalis; Escherichia coli; Lactobacillus thermophilus; Lactobacillus acidophilus; Lactobacillus brevis; Lactobacillus bulgaricus; Lactobacillus casei; Lactobacillus delbrueckii; Lactobacillus subspecies; Lactobacillus bulgaricus; Lactobacillus farciminis; Lactobacillus fermentum; Lactobacillus gallinarum; Lactobacillus jensenii; Lactobacillus paracasei; Lactobacillus plantarum; Lactobacillus reuteri; Lactobacillus rhamnosus; Lactobacillus lactis; Lactobacillus salivarius; Lactobacillus sobrius; Megasphaera elsdenii; Pediococcus acidolactici; Propionibacterium shermanii; Propionibacterium freudenreichii; Propionibacterium acidipropionici; Propionibacterium jensenii; Saccharomyces bourlrdii; Saccharomyces cerevisiae; Saccharomyces servisia; Streptococcus faecalis; Streptococcus faecium; Streptococcus gallolyticus; Streptococcus salivarius; Streptococcus subsp.; Streptococcus thermophilus; Streptococcus bovis and combinations thereof.

31. The method of claim 1, wherein said composition comprises a mixture of organisms, wherein said mixture of organisms is optionally a syntrophic mixture showing a syntrophic behavior.

32-33. (canceled)

34. The method of claim 31, wherein said mixture of organisms comprises at least one CO₂-utilizing organism.

35. The method of claim 34, wherein said CO₂-utilizing organism is an acetogen.

36. The method of claim 35, wherein said acetogen is selected from the group consisting of Acetitomaculum; Acetoanaerobium; Acetobacterium; Acetohalobium; Acetoneme; Bacillus; Blautia; Bryantella; Butyribacterium; Caloramator; Clostridium; Desulfovibrio; Entrococcus; Eubacterium; Gottschalkia; Holophage; Methylobacterium; Micrococcus; Moorella; Mycobacterium; Natronielle; Natronincola; Oxobacter; Peptoniphilus; Proteus; Reticulitermes; Rhizobium; Ruminococcus; Saccharomyces; Sinorhizobium; Sphingomonas; Sporomusa; Syntrophococcus; Thermoacetogenium; Tindallia; Treponema; Veillonella and combinations thereof.

37-39. (canceled)

40. The method of claim 1, wherein said applying improves hatchability of eggs.

41. The method of claim 1, wherein said applying reduces the time until hatching.

42. The method of claim 1, wherein said applying allows eggs to be stored for greater than two weeks with less than 15% reduction in the hatchability.
